(19) United States
(12) Patent Application Publication
(10) Pub. No.: US 2012/0323325 A1
(43) Pub. Date: Dec. 20, 2012

(54) AUTOLOGOUS IN SITU TISSUE ENGINEERING

(76) Inventor: Judith A. Fulton, (US)

(21) Appl. No.: 13/161,978

(22) Filed: Jun. 16, 2011

(51) Int. Cl. A61F 2/10; A61B 17/50

(52) U.S. Cl. 623/15.12; 606/131

(57) ABSTRACT

A kit that may be used to process autologous skin tissue that is grafted to the patient's wound for in situ growth of new skin tissue may include: a skin collection packet, a skin processing packet, a skin tissue application packet, and a wound preparation packet.

### I. BACKGROUND

A. Field of Invention

This invention relates generally to methods and apparatuses related to wound treatment and more specifically to methods and apparatuses related to processing autologous skin tissue that is grafted to a patient's wound for in situ growth of new skin tissue.

B. Description of the Related Art

Many wounds are difficult or impossible to treat due to the nature of the wound or the surrounding environment. For example, chronic wounds or burns require interventions that have varied success rates. Although tissue engineered products tend to be effective, their shortcomings include: expense, long preparation time, and short shelf life. However, battlefield situations, remote locales and third world poverty prevent the practicality of high tech treatments.

What is needed is an inexpensive, easy to use, self-contained kit which can be used at the bedside of a patient for processing autologous skin tissue that is grafted to the patient's wound for in situ growth of new skin tissue.

### II. SUMMARY

According to one embodiment of this invention, a kit may comprise: a skin collection packet comprising: (1) a first cutting tool that is suitable to remove an associated skin sample from an associated donor site; (2) forceps; (3) a first container that is suitable to hold the associated skin sample; and, (4) medical dressing material that is suitable to dress the donor site; a skin processing packet comprising; (1) a second cutting tool that is suitable to cut the associated skin sample into smaller pieces; (2) a second container; and, (3) a first mixing chamber suitable to receive the pieces of skin sample and skin processing solution for mixing; a skin tissue application packet comprising: (1) a filter suitable to filter the contents of the first mixing chamber after mixing; (2) a third container that is a supernatant discard tube; (3) a stirrer; (4) a fourth container containing a matrix that comprises at least one of collagen, a reversible thermogeling polymer, and fibrinogen, wherein the fourth container is suitable to mix the processed skin sample remaining in the filter and the matrix to obtain a suspension; and, (5) an application syringe that is operable to apply the suspension onto an associated wound site; and, a wound preparation packet comprising: (1) a debrider suitable to debride the associated wound site; and, (2) medical dressing material that is suitable to dress the associated wound site.

According to another embodiment of this invention, a method may comprise the steps of: (A) removing an autologous skin sample from a donor site; (B) mixing the skin sample with a matrix that comprises at least one of collagen, a reversible thermogeling polymer, and fibrinogen to create a skin tissue suspension; (C) placing the skin tissue suspension into an applicator; and, (D) using the applicator to simultaneously apply the skin tissue suspension and thrombin to a wound site. The method may further comprise at least one of the following steps: (1) treating the tissue suspension with cold plasma; and, (2) treating the wound site with cold plasma.

According to yet another embodiment of this invention, an apparatus may comprise: a skin sample removal device, comprising: (1) a housing; (2) a cutting tool that is suitable to remove an associated skin sample from a donor site and that is attached to the housing; (3) a grinder that is suitable to grind the associated skin sample and that is attached to the housing; and, (4) a first container that is attachable to and detachable from the housing; and, a skin tissue processing device, comprising: (1) a housing; (2) first, second, and third ports formed in the housing; (3) a first mixing chamber; and, (4) a first material moving device attached to the housing. The skin sample remove device may be operable to: (1) remove an associated skin sample from a donor site with the cutting tool; (2) grind the associated skin sample with the grinder; and, (3) hold the associated skin sample after grinding in the first container. The skin tissue processing device may be operable to: (1) receive for attachment the first container at the first port; (2) receive the associated skin sample through the first port; (3) receive the associated skin sample and an associated matrix forming a matrix mix through the second port and into the first mixing chamber to form a wound treatment solution; and, (4) apply the wound treatment solution from the first mixing chamber to an associated wound site with the first material moving device through the third port.

On advantage of this invention is that wounds requiring an autologous skin graft can be treated in remote locales.

Another advantage of this invention is that a kit may be self-contained and easily used.

Another advantage of this invention is that a much smaller amount of donor skin is required than used in standard grafts.

Yet another advantage of this invention is that cold plasma may be used to enhance migration and proliferation of skin cells and to kill micro-organisms.

Still another advantage of this invention is that a reversible thermogelling polymer may be used to provide for the use of a semi-solid gel at room and body temperatures.

Other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 is a block diagram illustrating a kit according to one embodiment of this invention.

FIG. 2 is a block diagram illustrating a skin tissue collection packet according to one embodiment of this invention.

FIG. 3 is a block diagram illustrating a skin tissue processing packet according to one embodiment of this invention.

FIG. 4 is a perspective view of a dissociator.

FIG. 5 is a block diagram illustrating a skin tissue application packet according to one embodiment of this invention.

FIG. 6 is a block diagram illustrating a wound preparation packet according to one embodiment of this invention.

FIG. 7 is a perspective view of a skin sample removal device according to one embodiment of this invention.

FIG. 8 is a perspective view of a skin tissue processing device according to one embodiment of this invention.

FIG. 9 is a perspective view of a punch biopsy array.

FIG. 10 is a bottom view of the punch biopsy array shown in FIG. 9.

FIG. 11 is a close up view of one of the punches shown in FIG. 9.

FIG. 12 is a close up view of one of the needles shown in FIG. 9.

FIG. 13 illustrates the application of cold plasma to a cell suspension.

### IV. DETAILED DESCRIPTION OF THE INVENTION

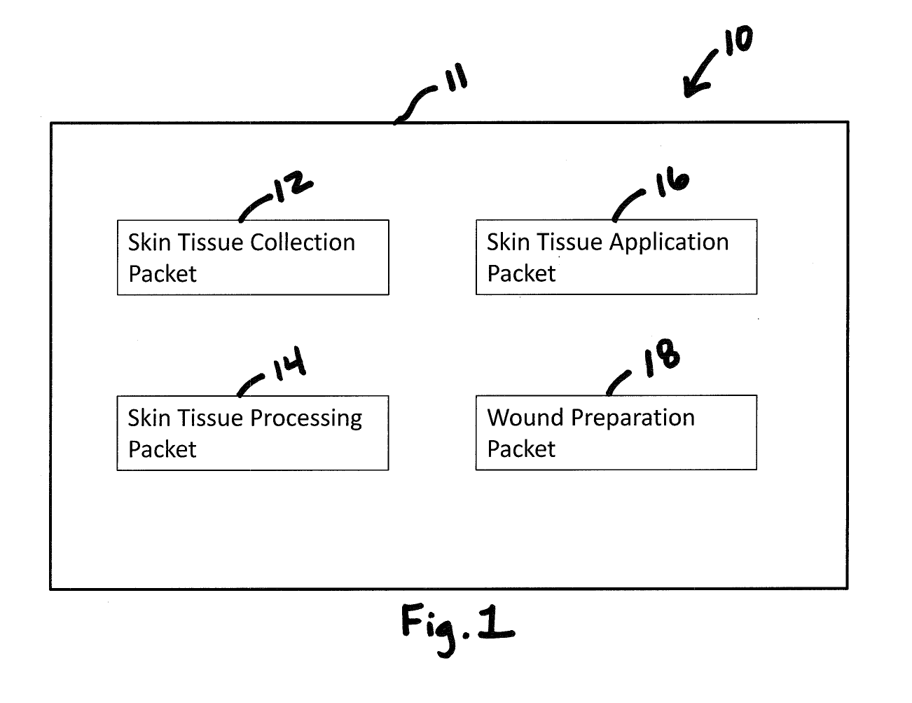

Referring now to the drawings wherein the showings are for purposes of illustrating embodiments of the invention only and not for purposes of limiting the same, and wherein like reference numerals are understood to refer to like components, FIG. 1 illustrates a kit 10 that may be used to treat a wound requiring a skin graft according to one embodiment of this invention. The kit 10 may be self-contained and may come packaged in a box, a bag or other such container 11 for easy transport. The kit 10 may include a skin tissue collection packet 12, a skin tissue processing packet 14, a skin tissue application packet 16, and, a wound preparation packet 18.

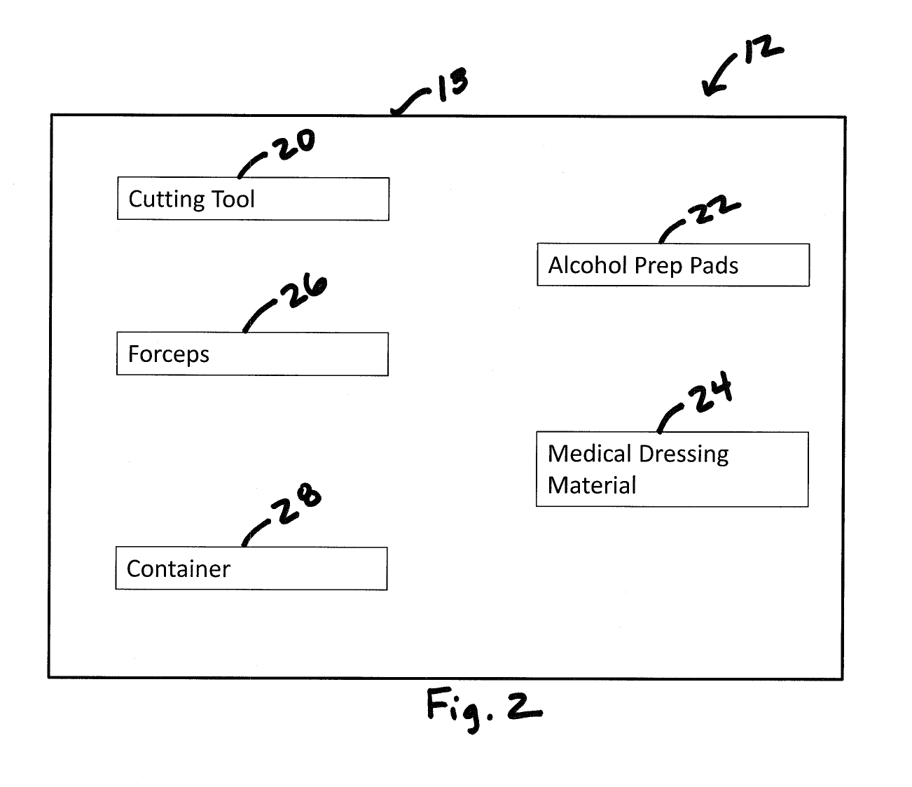

With reference now to FIGS. 1 and 2, the skin tissue collection packet 12 may be self-contained and may come packaged in a box, a bag or other such container 13 for easy transport. In one non-limiting embodiment the skin tissue collection packet 12 comes in a vacuum sealed bag. The skin tissue collection packet 12 may include the supplies that are needed to harvest a skin sample from a donor site on the patient. The supplies may include a cutting tool 20 that is suitable to remove the skin sample from the donor site. The cutting tool 20 used can be any chosen with the sound judgment of a person of skill in the art. In one embodiment, the cutting tool 20 is a dermatome. In another embodiment, the cutting tool 20 is part of a skin sample removal device 60 (shown in FIG. 7), which will be described below. The cutting tool 20 may be used to harvest a split-thickness skin sample of healthy tissue of minimal size. The skin tissue collection packet 12 may also include forceps 26 (or other such tool) for use in picking up the skin sample and a container 28 that is suitable to hold the skin sample. The container 28 may come pre-filled with a rinse solution such as phosphate buffered saline (PBS). In one embodiment, discussed further below, the container 28 is part of the skin sample removal device 60. The skin tissue collection packet 12 may also include alcohol prep pads 22 for use in cleaning and sterilizing the donor site and medical dressing material 24 for use in dressing the donor site after the skin tissue has been removed.

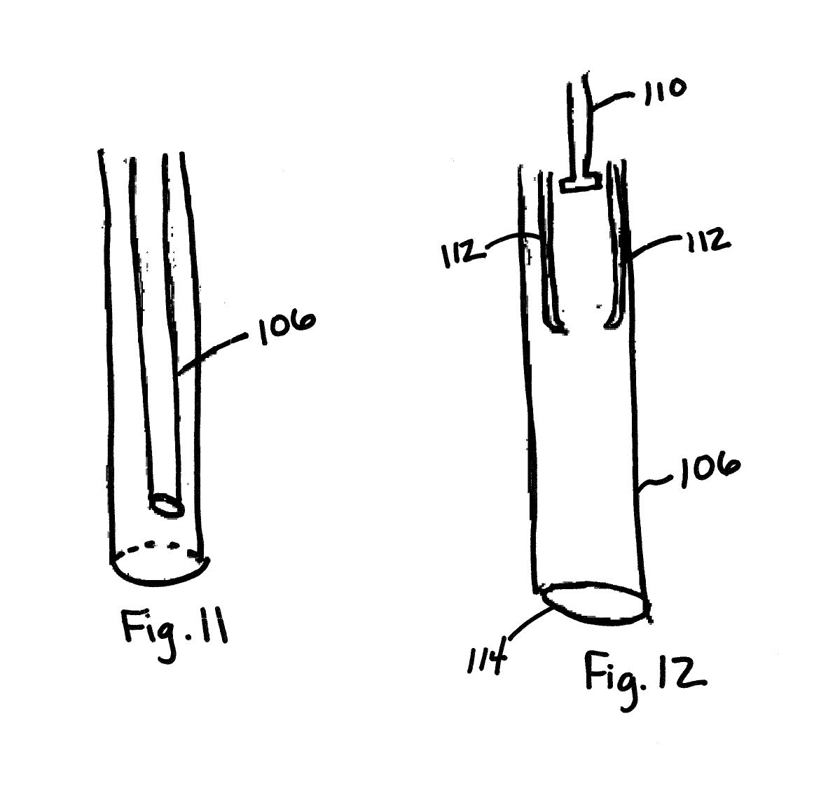

With reference now to FIGS. 2 and 9-12, in yet another embodiment, the cutting tool 20 is known as a punch biopsy array 100. The punch biopsy array 100 may include a housing 102 having multiple shafts or punches 104 positioned within the housing 102. As shown in FIG. 11, a retractable needle 106 may be positioned within each shaft 104. A spring (not shown) may be used to release each needle from within the shaft 104 to puncture the skin at the donor site a predetermined depth. A trigger 108 may be used to activate the springs and thus release the shafts 104. As shown in FIG. 12, each needle 106 may include a sharp edge 114 at its distal end and a plunger 110 that is movable within a pair of prongs 112. When the sharp edge 114 penetrates the skin, the prongs 112 open then close tightly around the core of skin within the needle 106. In this way, the skin core is lifted above the surface of intact skin and then can be cut away from the skin. The plunger 110 is used to push the core of skin out of the needle 106 and into the container 28. In use, the punch biopsy array 100 is placed on a clean donor site. The trigger 108 is activated and the array of needles 106 are driven (punched) a predetermined depth into the donor site. Each needle 106 collects a skin core as described. The skin cores are then simultaneously pushed out of the needles 106 and into the container 28. If necessary, the punch biopsy array 100 can be used more than once to collect the necessary amount of skin. When the punch biopsy array 100 is used, the skin tissue collection packet 12 may not require forceps 26.

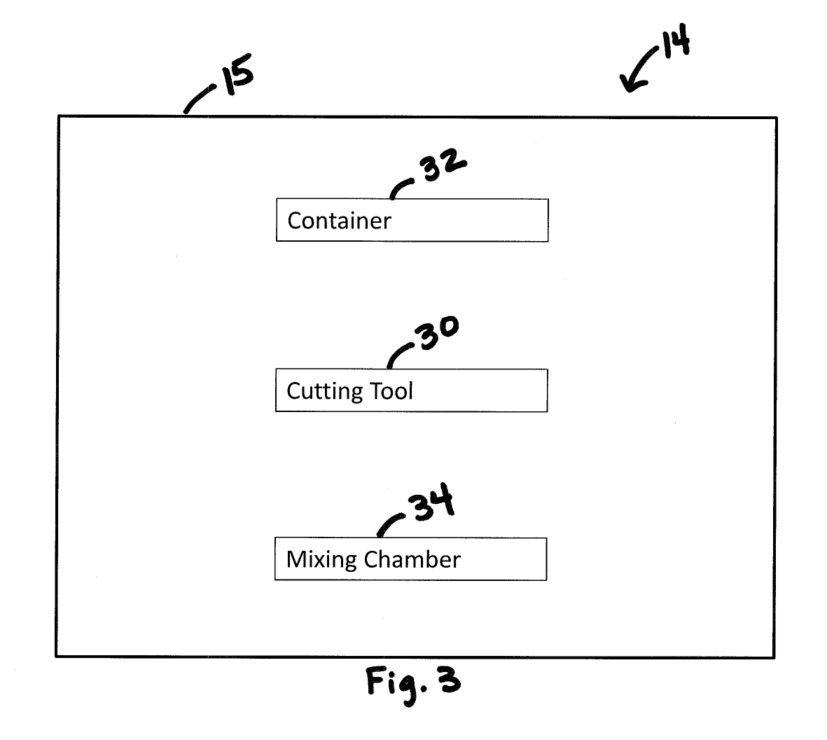

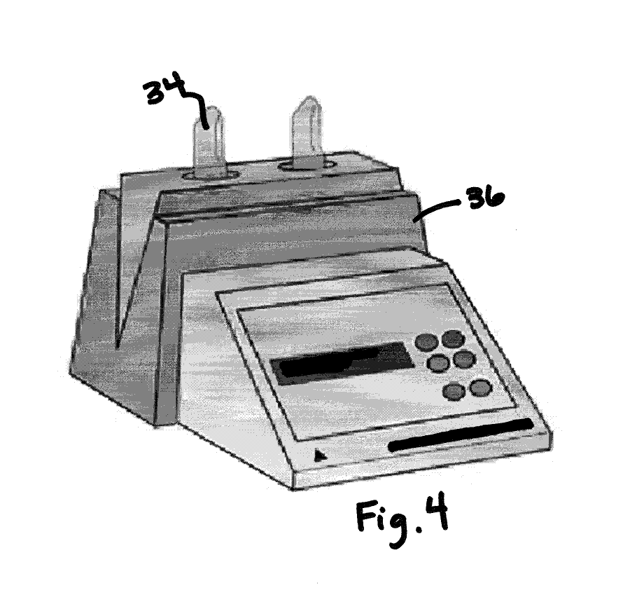

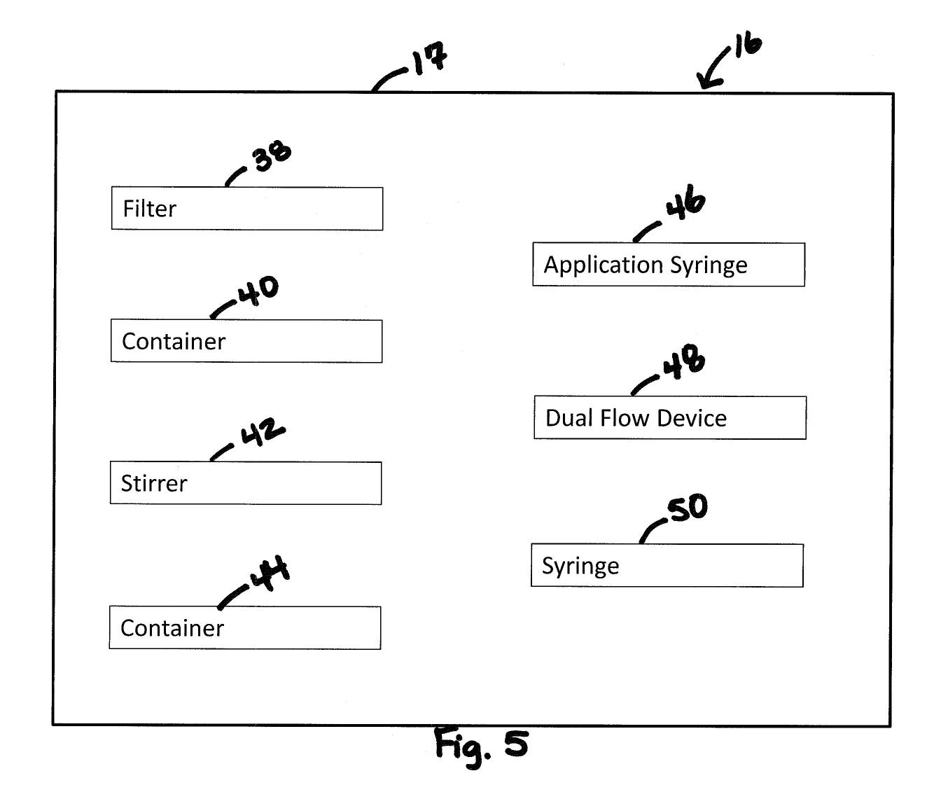

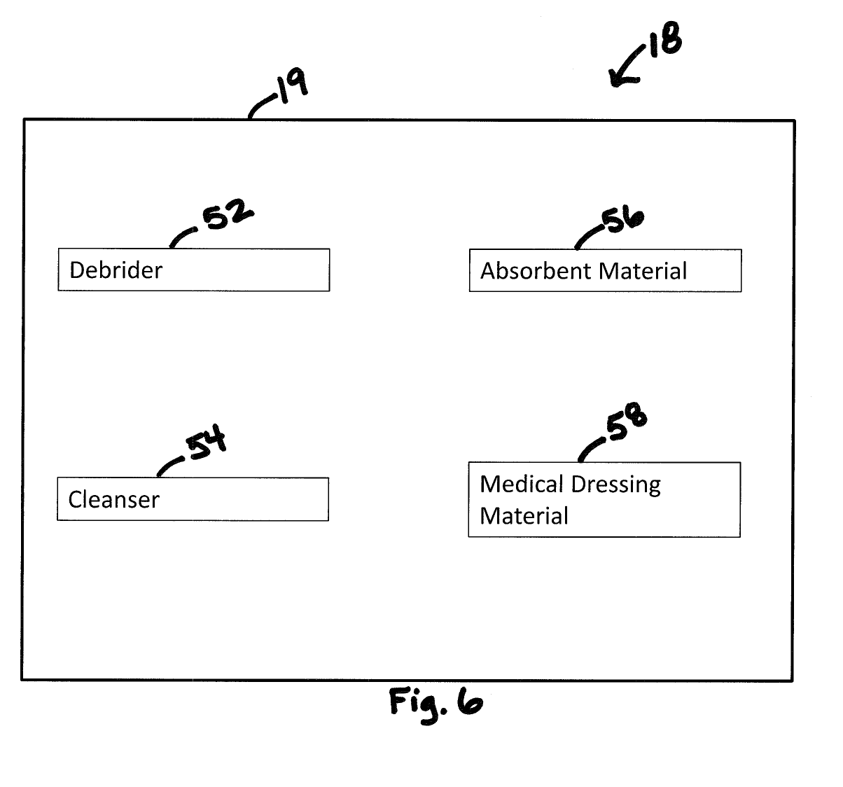

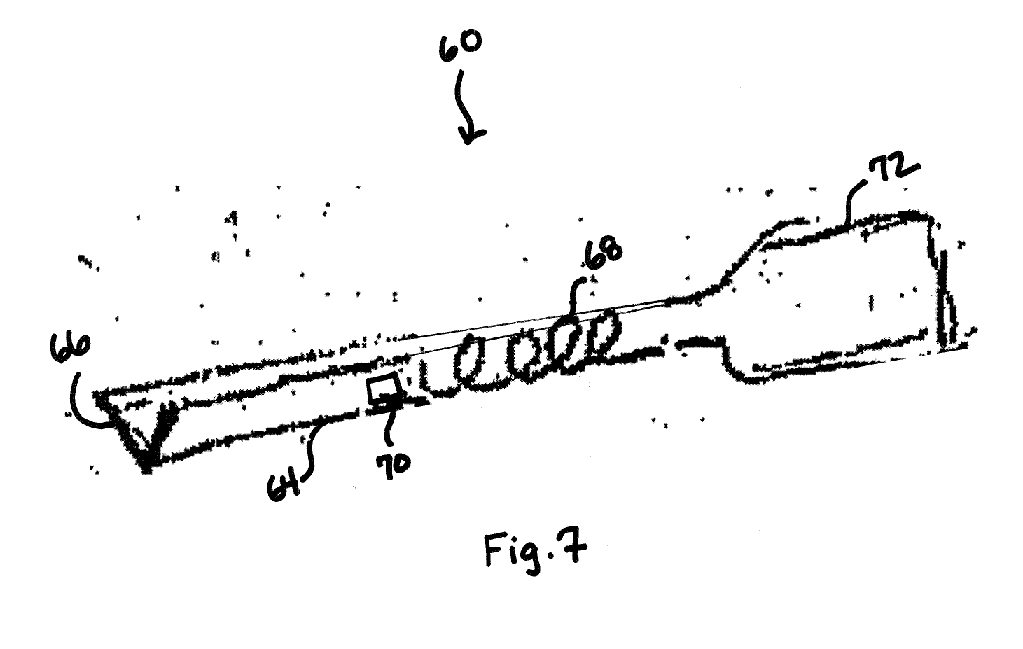

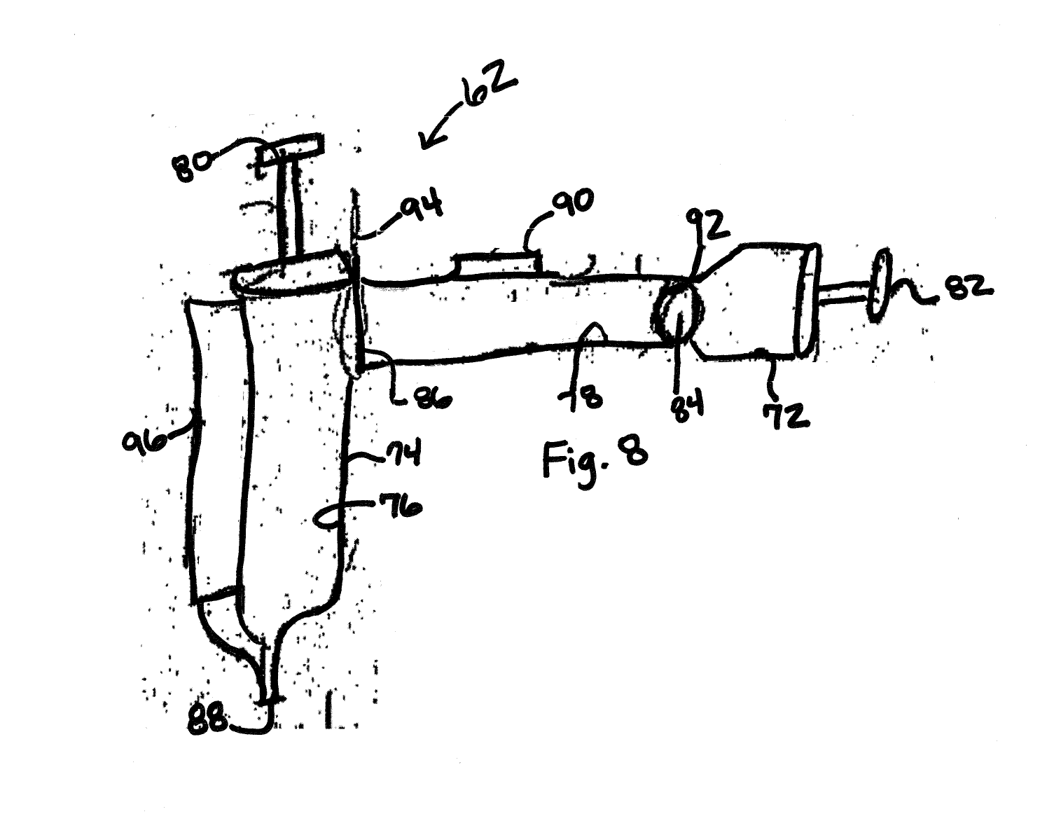

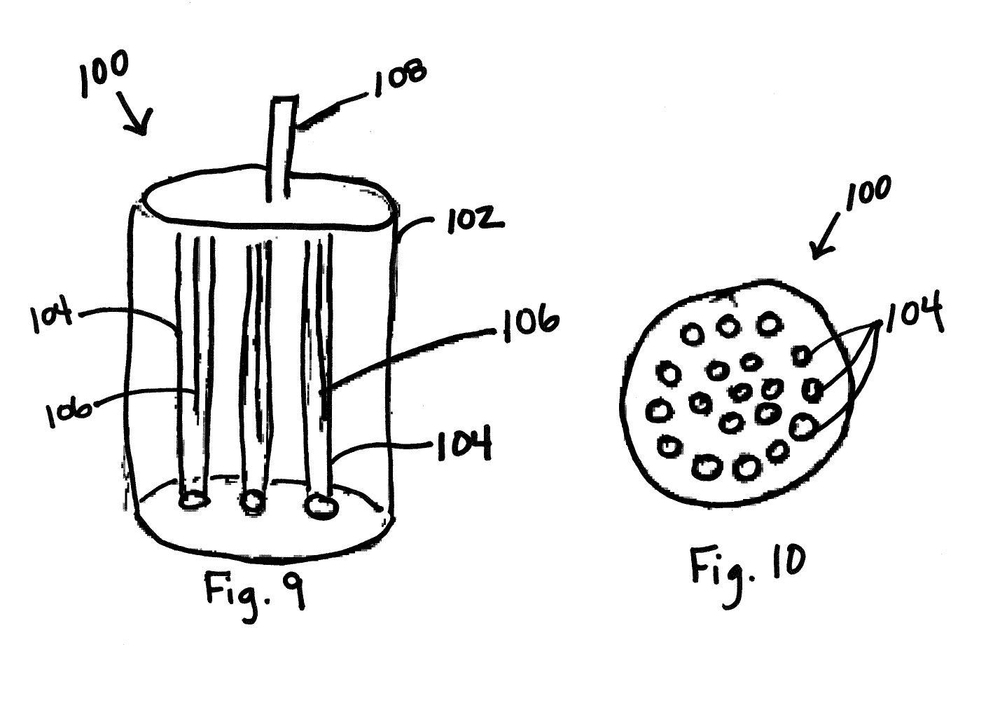

With reference now to FIGS. 1 and 3, the skin tissue processing packet 14 may be self-contained and may come packaged in a box, a bag or other such container 15 for easy transport. In one non-limiting embodiment the skin tissue processing packet 14 comes in a vacuum sealed bag. The skin tissue processing packet 14 may include the supplies that are needed to process the autologous skin sample. The skin tissue processing packet 14 may include a container 32, such as a petri dish, into which the skin sample is placed with the forceps 26. The skin tissue processing packet 14 may include a cutting tool 30, such as scissors, which is suitable to cut the skin sample into pieces and a mixing chamber 34. The mixing chamber 34 may be pre-filled with a skin processing solution such as Dulbecco's Modified Eagle Medium (DMEM). The pieces of the skin sample may be placed into the mixing chamber 34 where they are then mixed with the skin processing solution. In one embodiment, shown in FIG. 4, the mixing chamber 34 is a tube that is placed into a dissociator 36 and processed. For this embodiment, the kit 10 also includes the dissociator 36 which may or may not be placed within the skin tissue processing packet 14. The dissociator 36 may be battery powered and thus is well suited for use in an environment where electric power is not available.

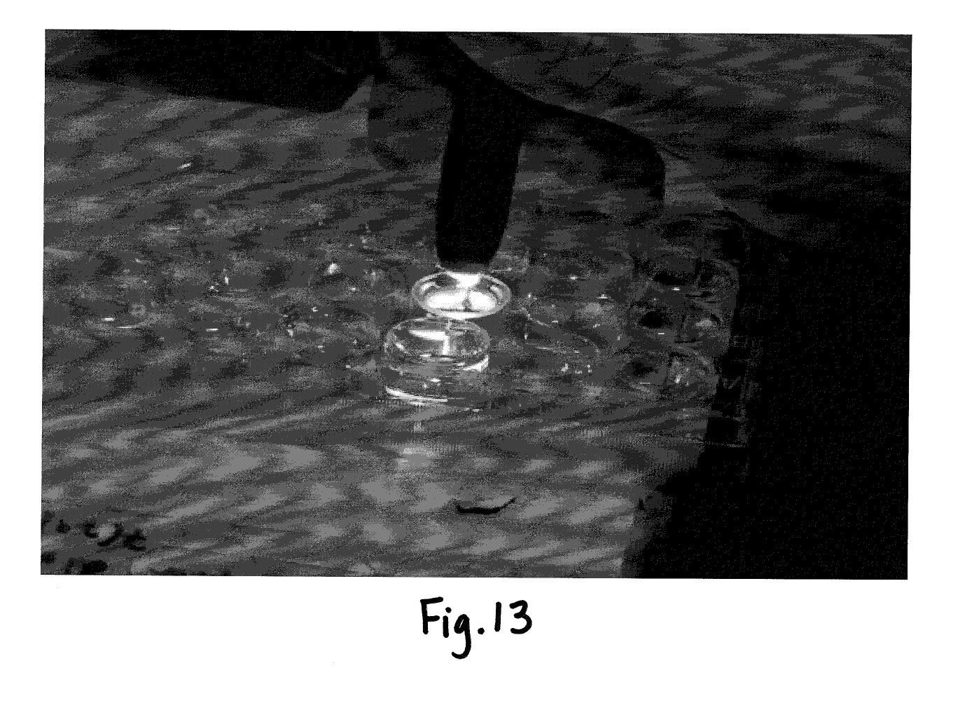

With reference now to FIGS. 1 and 5, the skin tissue application packet 16 may be self-contained and may come packaged in a box, a bag or other such container 17 for easy transport. In one non-limiting embodiment, the skin tissue application packet 16 comes in a vacuum sealed bag. The skin tissue application packet 16 may include the supplies that are needed to prepare the processed skin sample for application to the wound site. The skin tissue application packet 16 may include a filter 38 that is placed over a container 40 and a stirrer 42. The contents from the mixing chamber 34 are then filtered and stirred to remove the maximum amount of liquid. The container 40 may be a supernatant discard tube. In one embodiment, the skin sample is treated with cold plasma at this point. Cold plasma has been shown to have bactericidal properties and to enhance the activities (migration and proliferation) of skin cells. FIG. 13 illustrates the application of cold plasma to a cell suspension. The skin tissue application packet 16 may include another container 44 that is pre-filled with a matrix that comprises at least one of collagen, a reversible thermogeling polymer, and fibrinogen. The matrix may also include extra cellular matrix materials and/or growth factors.

With continuing reference to FIGS. 1 and 5, in one embodiment, the processed skin sample remaining in the filter and the matrix may then be mixed in the container 44 to obtain a uniform suspension. The suspension may be treated with cold plasma. The suspension may then be applied to the wound with a dual flow device 48. The suspension may be inserted into one container and thrombin into another. When the dual flow device 48 is activated, the suspension mixes with the thrombin to create a gel that is then applied to the wound site.

Still referring to FIGS. 1 and 5, in another embodiment, the matrix may include a reversible thermogelling polymer. Thermogelling polymers are in the liquid phase when kept cold and become semi-solid gels at room and body temperature. One non-limiting example of a thermogelling polymer is Pluronic® F 127 by BASF, The Chemical Company, a polyoxyethylene-polyoxypropylene triblock copolymer that has been used as a pharmaceutical vehicle for drug delivery. The processed skin sample remaining in the filter and the matrix may then be mixed in the container 44 to obtain a uniform suspension. The suspension may be treated with cold plasma. If a thermogelling polymer is used, after preparation of the tissue, the gel is chilled (such as: in a refrigerator, on ice, or with a cold pack that is activated upon breaking the inner ampule to initiate an endothermal reaction) to create the liquid in which the tissue will be suspended. The skin tissue application packet 16 may include an application syringe 46. The syringe 46 may be kept cool in order to keep the suspension liquid for easier application. The suspension is transferred to the application syringe 46 and the syringe 46 is used to apply the suspension to the wound site. If a thermogelling polymer is used, the suspension will gel as it is applied to the wound and reaches body temperature. The syringe 46 can be of any type chosen with the sound judgment of a person of skill in the art.

With reference now to FIGS. 1 and 6, the wound preparation packet 18 may be self-contained and may come packaged in a box, a bag or other such container 19 for easy transport. In one non-limiting embodiment the wound preparation packet 18 comes in a vacuum sealed bag. The wound preparation packet 18 may include the supplies that are needed to prepare the wound for tissue application (for skin grafting). The wound preparation packet 18 may include a debrider 52 suitable to debride the wound site, cleanser 54 suitable to clean the wound site, absorbent material 56 to remove excess moisture from the wound site, and medical dressing material 58 that is suitable to dress the wound site. The contents from the syringes 46, 50 may be applied to the cleaned wound and the contents may be allowed to gel within the wound cavity, thus filling the wound with matrix plus “micro grafts.” The wound may be treated with cold plasma at this point. The medical dressing material 58 is then applied to the wound site.

With reference now to FIGS. 7 and 8, a skin sample removal device 60 and a skin tissue processing device 62 may be used to treat a wound requiring a skin graft according to another embodiment of this invention. The skin sample removal device 60 and the skin tissue processing device 62 may, in one embodiment, be part of the kit 10. The skin sample removal device 60 may include a housing 64 and a cutting tool 66 attached to the housing 64 that is suitable to remove a skin sample from a donor site. In one embodiment, shown, the cutting tool 66 is a cutting blade attached to one end of the housing 64. The skin sample removal device 60 may include a grinder 68 that is attached to the housing 64 and that is suitable to grind the skin sample. In one embodiment, the grinder 68 is a corkscrew grinder. A motor 70 may be attached to the housing 64 and used to rotate the grinder 68 and/or to move the skin sample through the housing 64. In one embodiment, the motor 70 may be battery operated or mechanically driven and thus is well suited for use in an environment where electric power is not available. The skin sample removal device 60 may also include a container 72 that holds the skin sample after it has been ground. In one embodiment, the container 72 is attachable to and detachable from the housing 64. In one specific embodiment, the container 72 may have threads received in corresponding threads on the housing 64. In this case, the container 72 can be easily “unscrewed” from the housing 64 after the ground skin sample has been placed within the container 72.

With reference now to FIG. 8, the skin tissue processing device 62 may include, in one embodiment, a housing 74 having first and second mixing chambers 76, 78 and first and second material moving devices 80, 82. The second mixing chamber 78 may be pre-filled with a matrix as described above. Thus, the matrix may comprise at least one of collagen and fibrinogen, it may also include extra cellular matrix materials and/or growth factors and, in another embodiment, it may include a reversible thermogelling polymer. A first port 84 may be formed on the second mixing chamber 78, as shown. In use, the container 72 filled with the ground skin sample, may be removed from the skin sample removal device 60 and attached to the skin processing device 62 via the first port 84. In one embodiment, a perforate-able seal 92 is positioned over the first port 84. In this way sterilization of the skin tissue processing device 62 and the ground skin sample is maintained.

With reference now to FIGS. 7 and 8, the second material moving device 82 may be used to move the ground skin sample from the container 72 into the second mixing chamber 78. After mixing, the second material moving device 82 may also be used to move the ground skin sample mixed with the matrix from the second mixing chamber 78, through a second port 86, and into the first mixing chamber 76 to form a wound treatment solution. The second port 86 may be positioned between the first and second mixing chambers 76, 78, as shown. In one embodiment, a port closing device 94 is adjustable to close and open the second port 86. In one specific embodiment, the port closing device 94 may be a slidable gate that can be slid to seal the second port 86 and slid to open the second port 86. Once all materials are placed into the first mixing chamber 76, the port closing device 94 can be adjusted to close the second port 86 and the second mixing chamber 78, the container 72 and the second material moving device 82 can be removed from the skin tissue processing device 62. The first material moving device 80 can then be used to apply the wound treatment solution from the first mixing chamber 76 through a third port 88 and onto the wound site.

With reference now to FIG. 8, in one embodiment, the skin tissue processing device 62 may also include a container 96. The container 96 may be pre-filled with thrombin. For this embodiment, operation of the first material moving device 80 simultaneously applies the wound treatment solution from the first mixing chamber 76 and the thrombin from the container 96 to the associated wound site through the third port 88. The wound treatment solution may be allowed to gel within the wound cavity, thus filling the wound with matrix plus “micro grafts.” The wound may be treated with cold plasma at this point. In another embodiment, the skin tissue processing device 62 may include a fourth port 90 formed on the second mixing chamber 78. The fourth port 90 may be used to insert sterilization material into the second mixing chamber 78. In one embodiment, the first mixing chamber 76 is a first syringe and the second mixing chamber 78 is a second syringe. In this embodiment, the first material moving device 80 may be a first plunger and the second material moving device 82 may be a second plunger.

Numerous embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

I/we claim:

1. A kit comprising:
a skin collection packet comprising: (1) a first cutting tool that is suitable to remove an associated skin sample from an associated donor site; (2) a first container that is suitable to hold the associated skin sample; and, (3) medical dressing material that is suitable to dress the donor site;
a skin processing packet comprising; (1) a second cutting tool that is suitable to cut the associated skin sample into smaller pieces; (2) a second container; and, (3) a first mixing chamber suitable to receive the pieces of skin sample and skin processing solution for mixing;
a skin tissue application packet comprising: (1) a filter suitable to filter the contents of the first mixing chamber after mixing; (2) a third container that is a supernatant discard tube; (3) a stirrer; (4) a fourth container containing a matrix that comprises at least one of collagen and fibrinogen, wherein the fourth container is suitable to mix the processed skin sample remaining in the filter and the matrix to obtain a suspension; and, (5) an application syringe that is operable to apply the suspension onto an associated wound site; and,
a wound preparation packet comprising: (1) a debrider suitable to debride the associated wound site; and, (2) medical dressing material that is suitable to dress the associated wound site.

2. The kit of claim 1 wherein the first cutting tool is a punch biopsy array comprising:
a housing;
at least two punches positioned within the housing,
a retractable needle having a sharp edge at its distal end positioned within each punch;
a pair of prongs and a plunger positioned within each needle;
a trigger;
wherein the trigger is operable to drive the sharp edges of the retractable needles to penetrate the associated donor site,
wherein the pair of prongs are operable to obtain a skin core from the associated donor site; and,
wherein the plunger is operable to push the skin core out of the needle and into the first container.

3. The kit of claim 1 wherein wound preparation packet comprises cold plasma suitable to be used as a treatment for the associated wound site.

4. The kit of claim 1 wherein the matrix in the fourth container comprises a reversible thermogelling polymer.

5. The kit of claim 1 wherein:
the application syringe comprises a fifth container containing thrombin; and,
the application syringe is operable to apply the stirred contents from the fourth container and the thrombin onto the associated wound site.

6. The kit of claim 1 further comprising a battery powered dissociator.

7. A method comprising the steps of:
(A) removing an autologous skin sample from a donor site;
(B) mixing the skin sample with a matrix that comprises at least one of collagen and fibrinogen to create a skin tissue suspension;
(C) placing the skin tissue suspension into an applicator;
(D) using the applicator to simultaneously apply the skin tissue suspension and thrombin to a wound site;
wherein the method further comprises at least one of the following steps: (1) treating the tissue suspension with cold plasma; and, (2) treating the wound site with cold plasma.

8. The method of claim 7 wherein step (B) comprises the step of: mixing the skin sample with a matrix that comprises a reversible thermogelling polymer.

9. The method of claim 7 wherein after step (A) but before step (B) the method comprises the step of: grinding the skin sample with a grinder.

10. The method of claim 7 wherein after step (A) but before step (B) the method comprises the steps of:
placing the skin sample in a container that is attached to a skin sample removal device;
detaching the container from the skin sample removal device;
attaching the container to a skin tissue processing device;
moving the skin sample from the container into a mixing chamber that: (1) holds the matrix; and, (2) is mounted to the skin tissue processing device.

11. The method of claim 7 wherein step (A) comprises the step of: removing the autologous skin sample from the donor site with a dermatome.

12. The method of claim 7 wherein step (A) comprises the step of: removing the autologous skin sample from the donor site with a corkscrew grinder that comprises a cutting blade.

13. The method of claim 7 wherein step (D) comprises the step of: providing the applicator to comprise first and second syringes each comprising a plunger.

14. The method of claim 7 comprising all the steps of:
treating the tissue suspension with cold plasma; and,
treating the wound site with cold plasma.

15. An apparatus comprising:
a skin sample removal device, comprising: (1) a housing; (2) a cutting tool that is suitable to remove an associated skin sample from a donor site and that is attached to the housing; (3) a grinder that is suitable to grind the associated skin sample and that is attached to the housing; and, (4) a first container that is attachable to and detachable from the housing;
a skin tissue processing device, comprising: (1) a housing; (2) first, second, and third ports formed in the housing; (3) a first mixing chamber; and, (4) a first material moving device attached to the housing;
wherein the skin sample remove device is operable to: (1) remove an associated skin sample from a donor site with the cutting tool; (2) grind the associated skin sample with the grinder; and, (3) hold the associated skin sample after grinding in the first container; and,
wherein the skin tissue processing device is operable to: (1) receive for attachment the first container at the first port; (2) receive the associated skin sample through the first port; (3) receive the associated skin sample and an associated matrix forming a matrix mix through the second port and into the first mixing chamber to form a wound treatment solution; and, (4) apply the wound treatment solution from the first mixing chamber to an associated wound site with the first material moving device through the third port.

16. The apparatus of claim 15 wherein:
the skin tissue processing device further comprises a second mixing chamber and a second material moving device;
the first port is formed in the second mixing chamber;
the second port is formed between the first and second mixing chambers; and,
the second material moving device is operable to move the associated skin sample from the first container into the second mixing chamber and to move the matrix mix into the second mixing chamber.

17. The apparatus of claim 16 wherein the skin tissue processing device further comprises a fourth port formed on the second mixing chamber that is operable to receive an associated sterilization material.

18. The apparatus of claim 17 wherein:
a perforate-able seal is positioned over the first port; and,
a port closing device is adjustable to close the second port and to open the second port.

19. The apparatus of claim 18 wherein:
the skin tissue processing device further comprises a second container suitable to hold thrombin; and,
the first material moving device is operable to simultaneously apply the wound treatment solution from the first mixing chamber and the thrombin from the second container to the associated wound site through the third port.

20. The apparatus of claim 19 wherein:
the first mixing chamber is a first syringe;
the second mixing chamber is a second syringe;
the first material moving device is a first plunger; and,
the second material moving device is a second plunger.
